(19) United States
(12) Patent Application Publication
(10) Pub. No.: US 2010/0010212 A1
(43) Pub. Date: Jan. 14, 2010

(54) Processes for the preparation of (3R,4S)-4-((4-benzyloxy)phenyl)-1-(4-fluorophenyl)-3-((S)-3-(4-fluorophenyl)-3-hydroxypropyl)-2-azetidinone, an intermediate for the synthesis of ezetimibe

(76) Inventors: Vinod Kumar Kansal, Haryana (IN); Suhail Ahmad, New Delhi (IN); Bhupendra Tyagi, Uttaranchal (IN); Nitin Gupta, Uttar Pradesh (IN)

Correspondence Address: KENYON & KENYON LLP, ONE BROADWAY, NEW YORK, NY, 10004, US

(21) Appl. No.: 12/583,305

(22) Filed: Aug. 17, 2009

Related U.S. Application Data
(62) Division of application No. 11/517,704, filed on Sep. 8, 2006.
(60) Provisional application No. 60/715,919, filed on Sep. 8, 2005.
(60) Provisional application No. 60/832,430, filed on Jul. 20, 2006.

(51) Int. Cl. C07D 205/08; A61K 31/397

(52) U.S. Cl. 540/200; 514/210.02

(57) ABSTRACT

The invention encompasses (3R,4S)-4-((4-benzyloxy)phenyl)-1-(4-fluorophenyl)-3-(3-(4-fluorophenyl)-3-oxopropyl)-2-azetidinone (Compound 2a) having an enantiomeric purity of at least about 97.5%. The invention also encompasses Compound 2a having a chemical purity of at least about 97%. The invention further encompasses processes for preparing Compound 2a from Compound 1 having the following formula:

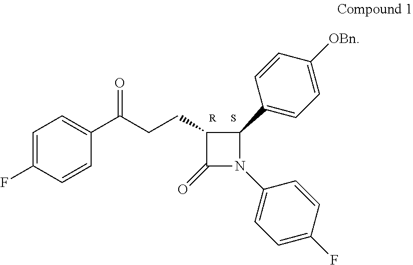

The invention also encompasses processes for preparing a compound having the following formula:

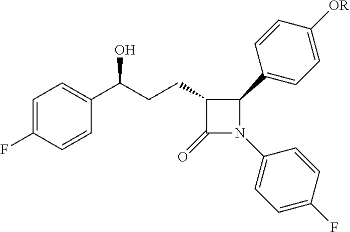

from a compound having the following formula:

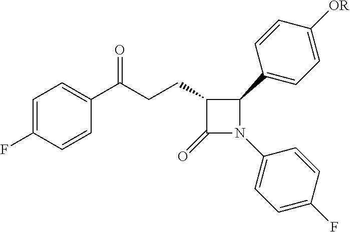

wherein R is selected from the group consisting of: H or a hydroxyl protecting group. The invention also encompasses processes for preparing Compound 2a, preferably to form Compound 2a-Form 01. Also included are processes for preparing ezetimibe from Compound 2a-Form 01 or Compound 2a prepared according to the invention, compositions containing such ezetimibe, and methods for reducing cholesterol using such compositions

### CROSS-REFERENCE TO RELATED APPLICATIONS

The present application claims the benefit of U.S. Provisional Patent Application No. 60/715,919, filed Sep. 8, 2005, and Provisional Patent Application No. 60/832,430, filed Jul. 20, 2006, the contents of each of which are incorporated herein by reference.

### FIELD OF THE INVENTION

The invention relates to the preparation of compounds for the synthesis of certain hydroxy-alkyl substituted azetidinones. More particularly, the invention relates to (3R,4S)-4-((4-benzyloxy)phenyl)-1-(4-fluorophenyl)-3-((S)-3-(4-fluorophenyl)-3-hydroxypropyl)-2-azetidinone, methods for its preparation, and methods for its use in the preparation of ezetimibe.

### BACKGROUND OF THE INVENTION

Hydroxy-alkyl substituted azetidinones are useful as hypercholesterolemia agents in the treatment and prevention of atherosclerosis. Ezetimibe, 1-(4-fluorophenyl)-3(R)-[3-(4-fluorophenyl)-3(S)-hydroxypropyl]-4(S)-(4-hydroxyphenyl)-2-azetidinone, is a selective inhibitor of intestinal cholesterol and related phytosterol absorption. The empirical formula for ezetimibe is C₂₄H₂₁F₂NO₃, and its molecular weight is 409.4. Ezetimibe is a white, crystalline powder that is freely to very soluble in ethanol, methanol, and acetone and practically insoluble in water. Ezetimibe has the following chemical structure:

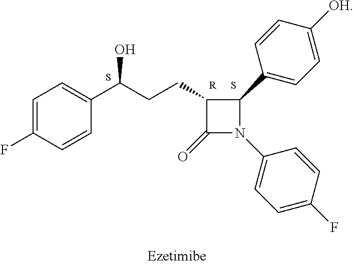

Ezetimibe is the active ingredient in ZETIA®, manufactured by Merck/Schering-Plough Pharmaceuticals, and is approved by the United States Food and Drug Administration for use in patients with high cholesterol to reduce LDL cholesterol and total cholesterol.

Ezetimibe can be prepared by reducing (3R,4S)-4-((4-benzyloxy)phenyl)-1-(4-fluorophenyl)-3-(3-(4-fluorophenyl)-3-oxopropyl)-2-azetidinone (Compound 1) with borane dimethyl sulfide complex or borane tetrahydrofuran complex in tetrahydrofuran in the presence of Corey's reagent and subsequently deprotecting the benzyl group, as shown in scheme 1, below.

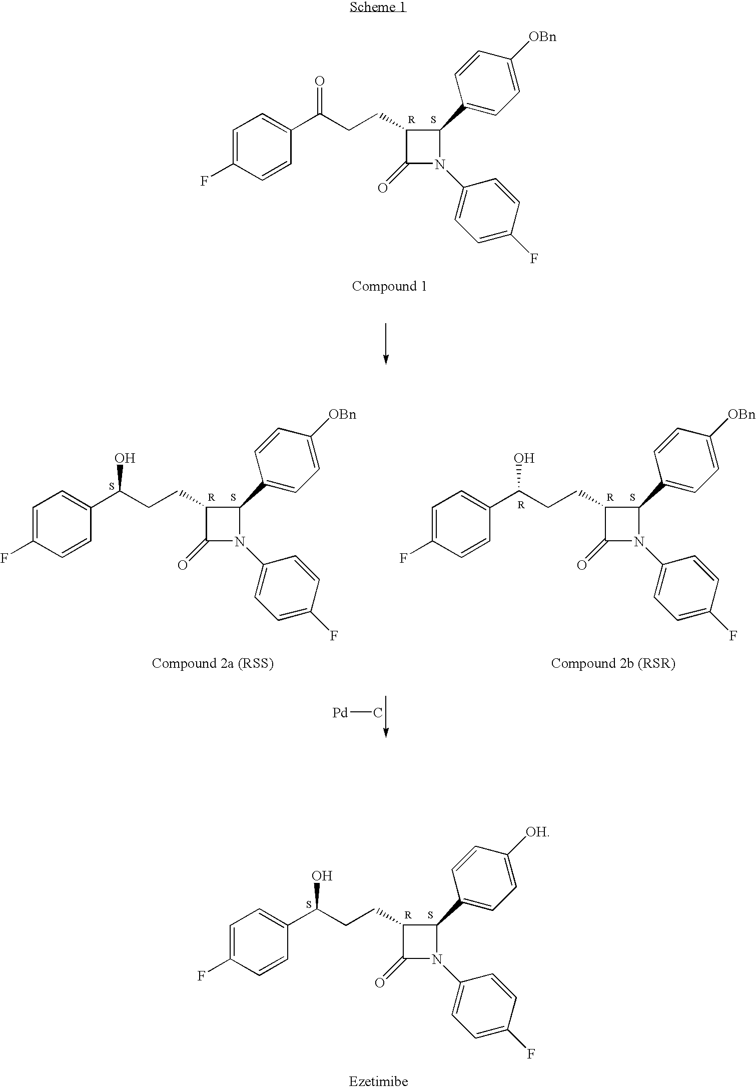

The reduction process produces two isomers, Compound 2a, or (3R,4S)-4-((4-benzyloxy)phenyl)-1-(4-fluorophenyl)-3-((S)-3-(4-fluorophenyl)-3-hydroxypropyl)-2-azetidinone, and Compound 2b, or (3R,4S)-4-((4-benzyloxy)phenyl)-1-(4-fluorophenyl) 3-((R)-3-(4-fluorophenyl)-3-hydroxypropyl)-2-azetidinone. Compound 2a is the desired isomer that produces ezetimibe of the proper chirality. Compound 2b is an undesirable isomer that is very difficult to remove both during reduction as well as the final synthesis to form ezetimibe. It has been reported that Compound 2b is typically produced in about 8 to 10% yield during the reduction process.

U.S. Pat. No. 5,886,171 (“the '171 patent”) reports that a crystalline form of Compound 2a is obtained from ethyl acetate-hexane solvent mixture. The crystalline form of Compound 2a disclosed in the '171 patent is denominated herein as Form 01.

There is a need for methods for preparing Compound 2a having low amount of the undesirable isomer Compound 2b.

### DESCRIPTION OF THE FIGURES

FIG. 1a: X-Ray Diffraction Pattern of Compound 2a-Form 01 recrystallized from toluene in accordance with Example 1.

FIG. 1b: X-Ray Diffraction Values of Compound 2a-Form 01 recrystallized from toluene in accordance with Example 1.

FIG. 2a: X-Ray Diffraction Pattern of Compound 2a-Form 01 crystallized from ethyl acetate-hexane reproduced from the '171 patent.

FIG. 3a: X-Ray Diffraction Pattern of Compound 2a-Form 01 recrystallized from toluene in accordance with Example 2.

FIG. 3b: X-Ray Diffraction Values of Compound 2a-Form 01 recrystallized from toluene in accordance with Example 2.

FIG. 4a: X-Ray Diffraction Pattern of Compound 2a-Form 01 recrystallized from ethanol in accordance with Example 3.

FIG. 4b: X-Ray Diffraction Values of Compound 2a-Form 01 recrystallized from ethanol in accordance with Example 3.

### SUMMARY OF THE INVENTION

In one embodiment, the invention encompasses Compound 2a having an enantiomeric purity of at least about 97.5%, preferably at least about 98.5%, and more preferably at least about 99%.

In another embodiment, the invention encompasses Compound 2a having less than about 2.5% Compound 2b, more preferably less than about 1.5%, and more preferably less than about 1% by area percent HPLC.

In one embodiment, the invention encompasses Compound 2a having a chemical purity of at least about 97%, preferably at least about 98%, and more preferably at least about 99% by area percent HPLC.

In one embodiment, the present invention encompasses a process for preparing Compound 2a comprising combining (3R,4S)-4-((4-benzyloxy)phenyl)-1-(4-fluorophenyl)-3-(3-(4-fluorophenyl)-3-oxopropyl)-2-azetidinone (Compound 1) with a solvent selected from the group consisting of cyclic ether, ether, halogenated hydrocarbons, aromatic hydrocarbons, and mixtures thereof to obtain a solution; adding an acid, a chiral catalyst, and a sufficient amount of a borane reducing agent to obtain Compound 2a; and recovering Compound 2a.

Preferably, the process produces Compound 2a having an enantiomeric purity of at least about 97.5%, more preferably at least about 98.5%, and most preferably at least about 99%.

In one embodiment, the invention encompasses a process for preparing Compound 2a comprising crystallizing Compound 2a from a solvent comprising isopropanol, ethanol, and mixtures thereof, using an antisolvent such as hexane or heptane. Preferably, the Compound 2a obtained is Compound 2a-Form 01.

The invention further encompasses a process for crystallizing Compound 2a comprising crystallizing Compound 2a from a solvent comprising toluene, ethanol, acetonitrile, methyl isobutyl ketone (MIBK), dichloromethane-hexane, methanol, acetone-water, and mixtures thereof. Preferably, the crystallized Compound 2a is Compound 2a-Form 01. Preferably, the process is carried out after a first crystallization step.

In another embodiment, the invention encompasses Compound 2a prepared according to a process of the invention.

In another embodiment, the invention encompasses a process for preparing ezetimibe comprising converting a Compound 2a of the invention to ezetimibe. The invention also encompasses a process for preparing ezetimibe comprising preparing Compound 2a according to a process of the invention, and converting Compound 2a to ezetimibe. The invention also encompasses ezetimibe prepared therefrom.

In another embodiment, the invention encompasses a process for preparing ezetimibe comprising preparing Compound 2a-Form 01 according to a process of the invention, and converting Compound 2a-Form 01 to ezetimibe. The invention also encompasses ezetimibe prepared therefrom.

In one embodiment, the invention encompasses a process for preparing a compound of the formula:

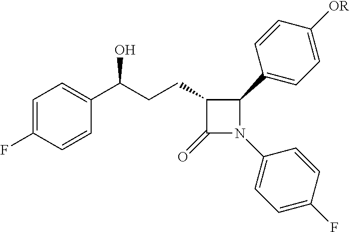

comprising combining a starting compound of the formula:

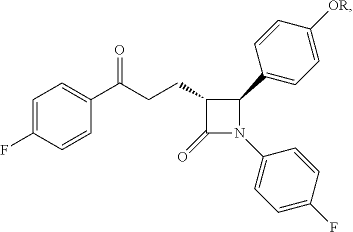

wherein R is H or a hydroxyl protecting group; a chiral catalyst; a hydrogen source including at least one of formic acid or a salt thereof, C₃-C₁₃ secondary alcohol, or cyclohexadiene; and an organic solvent, and recovering the product.

In another embodiment, the invention encompasses a process for preparing a compound of the formula:

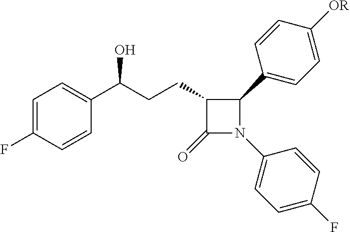

comprising combining a starting compound of the formula:

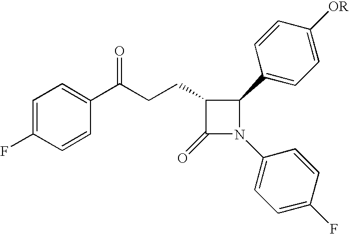

wherein R is H or a hydroxyl protecting group, and a chiral catalyst under an inert gas environment; adding an organic base to obtain a reaction mixture; subjecting the reaction mixture to a hydrogen pressure of about 4 bars to about 40 bars to produce the product; and recovering the product.

In one embodiment, the invention encompasses a process for preparing ezetimibe comprising preparing Compound 2a according to a process of the invention, and converting Compound 2a to ezetimibe. The invention also encompasses ezetimibe prepared from any one of the processes of the invention. The invention further encompasses a pharmaceutical composition comprising ezetimibe prepared according to a process of the present invention, and at least one pharmaceutically acceptable excipient.

In another embodiment, the invention encompasses a process for preparing a pharmaceutical formulation comprising combining ezetimibe prepared according a process of the invention with at least one pharmaceutically acceptable excipient.

In one embodiment, the invention encompasses the use of ezetimibe prepared according to a process of the present invention for the manufacture of a pharmaceutical composition.

In another embodiment, the invention encompasses a method of reducing cholesterol comprising administering to a mammal in need thereof a composition of the invention.

### DETAILED DESCRIPTION OF THE INVENTION

As used herein, the term “ezetimibe-ketone” refers to 1-(4-fluorophenyl)-3(R)-[3-(4-fluorophenyl)-3-oxopropyl]-4(S)-(4-hydroxyphenyl)-2-azetidinone.

The reduction of Compound 1 with borane reducing agents produces two isomers, Compound 2a and Compound 2b:

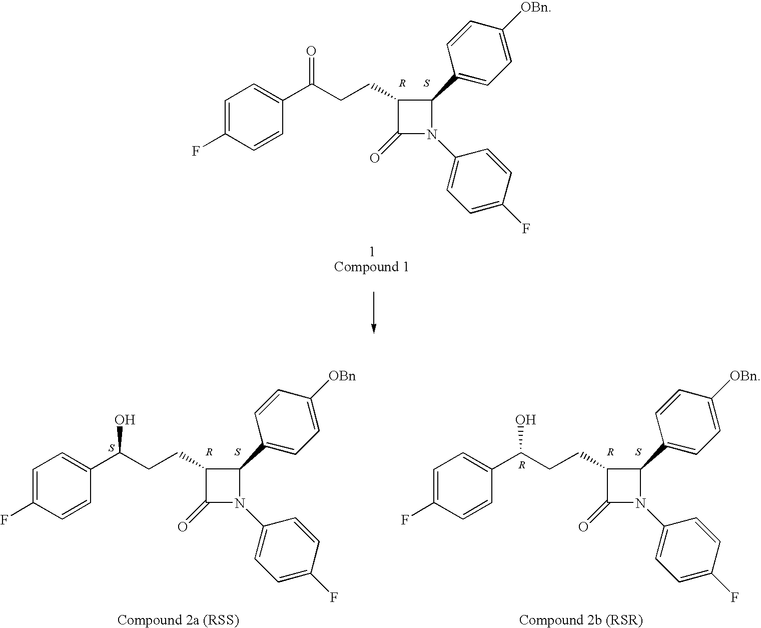

The invention encompasses Compound 2a having an enantiomeric purity of at least about 97.5%, more preferably at least about 98.5%, and most preferably at least about 99%.

The invention also encompasses Compound 2a having less than about 2.5% Compound 2b, more preferably less than about 1.5%, and more preferably less than about 1% by area percent HPLC.

The invention further encompasses Compound 2a having a chemical purity of at least about 97%, preferably at least about 98%, and more preferably at least about 99% by area percent HPLC.

The invention encompasses a process for preparing Compound 2a comprising: combining (3R,4S)-4-((4-benzyloxy)phenyl)-1-(4-fluorophenyl)-3-(3-(4-fluorophenyl)-3-oxopropyl)-2-azetidinone (Compound 1) with a solvent selected from the group consisting of cyclic ether, ether, halogenated hydrocarbons, aromatic hydrocarbons, and mixtures therefore to obtain a solution; adding an acid, a chiral catalyst, and a sufficient amount of a borane reducing agent to obtain Compound 2a; and recovering Compound 2a.

Preferred examples of cyclic ethers are substituted or unsubstituted C₂-C₁₀ ethers such as ethyleneoxide, tetrahydrofuran, 1-4-dioxane, 2-alkyl (e.g., C₁-C₆) tetrahydrofuran, and the like. C₄-C₆ cyclic ethers are preferred.

As used herein, the term “substituted” or “substituent” refers to moieties commonly known in the art. For example, an alkyl group, an alkenyl group, a cyclic alkyl group, an aralkyl group, a cyclic alkenyl group, a halogen atom, a nitro group, a cyan group, an aryl group, an alkoxy group, an aryloxy group, an alkoxycarbonyl group, an aryloxycarbonyl group, a sulfamoyl group, a carbamoyl group, an acylamino group, a diacylamino group, a ureido group, a urethane group, a sulfonamido group, an arylsulfonyl group, an alkylsulfonyl group, an alkylthio group, an arylthio group, an alkylamino group, a hydroxy group, a mercapto group, or the like. Of these substituents, those which are C₀-C₆ groups are preferred in some embodiments. In some embodiments, of these substituents, those which are C₀-C₂ groups are preferred.

Preferred examples of ethers are C₂-C₁₀ ethers such as diethyl ether, isopropyl ether, diisopropyl ether, methyl tert-butyl ether, and the like. C₄-C₆ ethers are preferred.

Preferred examples of aromatic hydrocarbons are substituted or unsubstituted C₆-C₁₀ aromatic hydrocarbons such as benzene, toluene, xylene, and the like. C₆-C₈ aromatic hydrocarbons are preferred.

Preferred examples of halogenated hydrocarbons are cyclic or acyclic, saturated or unsaturated, aliphatic or aromatic hydrocarbons. Examples of halogenated hydrocarbons include halogenated alkanes such as chloromethane, dichloromethane, chloroethane, dichlorotrifluoroethane, difluoroethane, hexachloroethane, or pentafluoroethane; halogenated alkenes such as such as tetrachloroethene, dichloroethene, trichloroethene, vinyl chloride, chloro-1,3-butadiene, or chlorotrifluoroethylene; or halogenated benzenes such as benzotrichloride, benzyl chloride, bromobenzene, chlorobenzene, chlorotoluene, dichlorobenzene, fluorobenzene, or trichlorobenzene. A preferred halogen is chlorine. Preferred halogenated hydrocarbons are aromatic hydrocarbons or C₁-C₄ alkanes, and more preferably chlorinated aromatic hydrocarbons or C₁-C₄ alkanes. More preferred halogenated hydrocarbons are chlorobenzene, o- or p-dichlorobenzene, dichloromethane, or o-chlorotoluene.

It is believed that addition of an acid with the borane reducing agent reduces the formation of the undesirable isomer Compound 2b (RSR configuration). Presence of an acid increases the enantiomeric purity of Compound 2a, which is a desired isomer useful for the preparation of ezetimibe (RSS configuration).

Preferably, the acid is selected from the group consisting of methanesulfonic acid, trifluoroacetic acid, boron trifluoride etherate, and mixtures thereof. The preferred acid is methanesulfonic acid.

Preferably, the ratio of acid to Compound 1 is in a molar % of about 1% to about 5%, more preferably about 1.6% to about 2%.

Preferably, the solvent includes at least one of tetrahydrofuran, toluene, dichloromethane, 2-methyl THF, THF-methyl tert butyl ether, or ethyl acetate.

Preferably, the chiral catalyst includes at least one of (R)-tetrahydro-1-methyl-3,3-diphenyl-1H,3H-pyrrolo[1,2-C][1,3,2]oxazaborolidine (“(R)-Me-CBS”), or (R)-tetrahydro-1-phenyl-3,3-diphenyl-1H,3H-pyrrolo[1,2-C][1,3,2]oxazaborolidine (“(R)-phenyl-CBS”). More preferably, the chiral catalyst is (R)-Me-CBS. Preferably, the chiral catalyst is added at a temperature of about 25° C. to about 30° C.

Preferably, the ratio of the chiral catalyst to Compound 1 is in a molar percentage of about 20% to about 40%, and more preferably, about 20% to about 35%.

Borane reducing agents include borane complexes such as borane-methyl sulfide complex, borane-morpholine complex, borane-pyridine complex, borane-tetrahydrofuran complex, borane-tributylphosphine complex, borane-triethylamine complex, borane-trimethylamine complex, borane-1,4 thioxane,

Preferably, the borane reducing agent is selected from the group consisting of borane complexes including borane-tetrahydrofuran complex or borane-dimethylsulfide complex, borane 1,4-dioxane, borane diethylaniline, borane N-ethyl-N-isopropylaniline, N-borane phenylamine, catecholborane, borane (preferably in situ generated borane), and mixtures thereof. More preferably, the borane reducing agent is a borane-tetrahydrofuran complex or a borane-dimethylsulfide complex.

As used herein, a “sufficient amount” of a borane reducing agent is an amount that will reduce Compound 1 to form Compound 2. In one embodiment the ratio of the borane reducing agent to Compound 1 is in a molar % of about 100% to about 200% (or about 1.0 to about 2.0 molar equivalent of Compound 1); in another embodiment the ratio is about 100% to about 170% (or about 1.0 to about 1.7 molar equivalent of Compound 1).

Preferably, the borane reducing agent is added after the acid and chiral catalyst, and more preferably after cooling. The borane reducing agent can be added before or after Compound 1. If the borane reducing agent is added before Compound 1, it is preferably added at a temperature of about −30° C. to about −15° C., and more preferably at a temperature of about −25° C. to about −20° C.

Preferably, prior to the recovery step a reaction mixture containing Compound 2a is obtained. Preferably, the reaction mixture is stirred. Preferably, the stirring is at a temperature of about 0° C. to about 15° C., more preferably about 10° C.

Preferably, the recovery step comprises quenching the reaction mixture with a solvent including at least one of methanol or acetone; and extracting it. Preferably, prior to the extraction, an acid suitable to decompose the excess borane complex, e.g., HCl, is added. Preferably, the reaction mixture is extracted with ethyl acetate and water. The organic layer is preferably washed, dried, for example over sodium sulfate, distilled and degassed, to produce Compound 2a.

Compound 2a may be crystallized in a crystallization solvent comprising isopropanol, ethanol, and mixtures thereof, preferably using an antisolvent such as hexane or heptane. Preferably, the solvent/antisolvent include isopropanol-heptane, ethanol-heptane, and mixtures thereof. Preferably, the isopropanol-heptane or ethanol-heptane ratio is from about 10:1 to about 1:10 by volume, and more preferably about 1:5 by volume. Preferably, an antisolvent including at least one of n-heptane or n-hexane is used.

Preferably, the crystallized Compound 2a has an enantiomeric purity of at least about 97.5%, more preferably at least about 98.5%, and most preferably at least about 99%. Preferably, the crystallized Compound 2a is Compound 2a-Form 01.

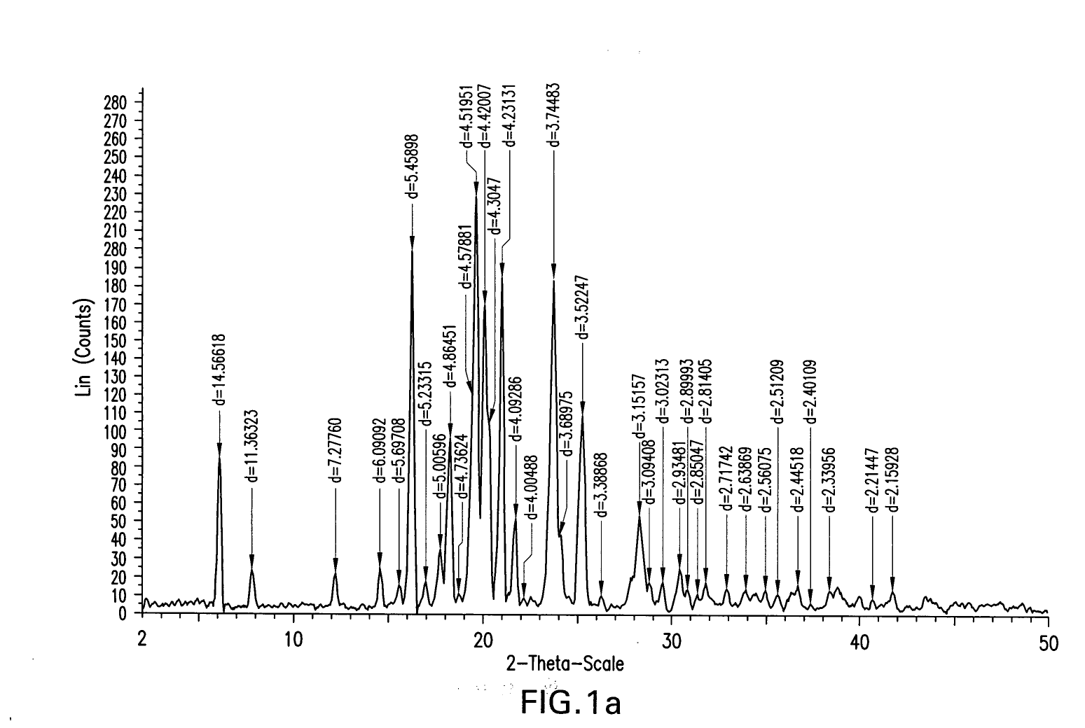

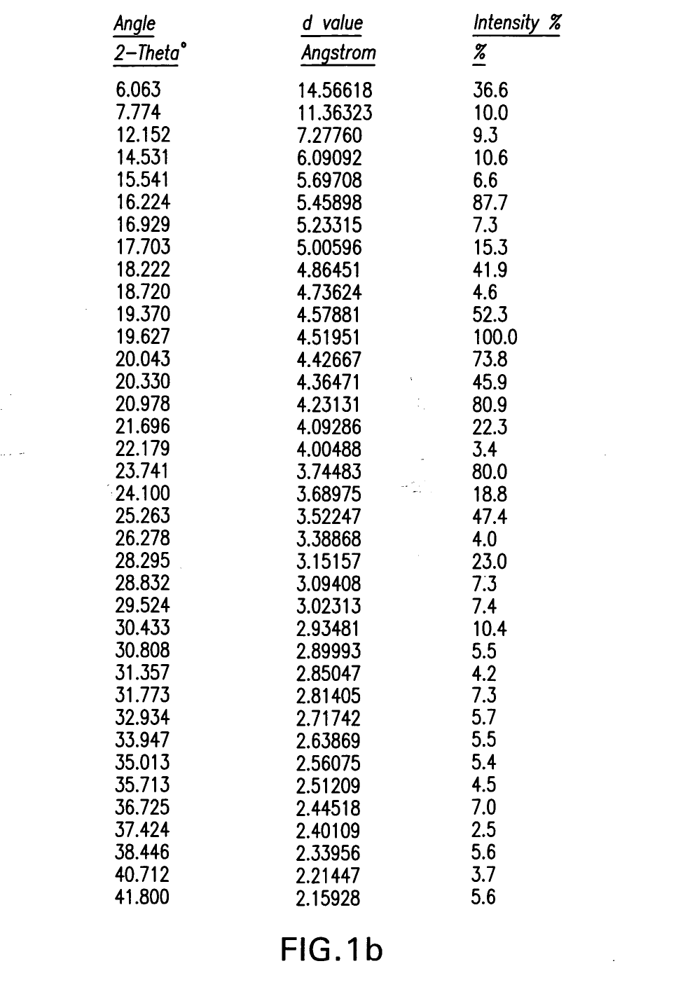

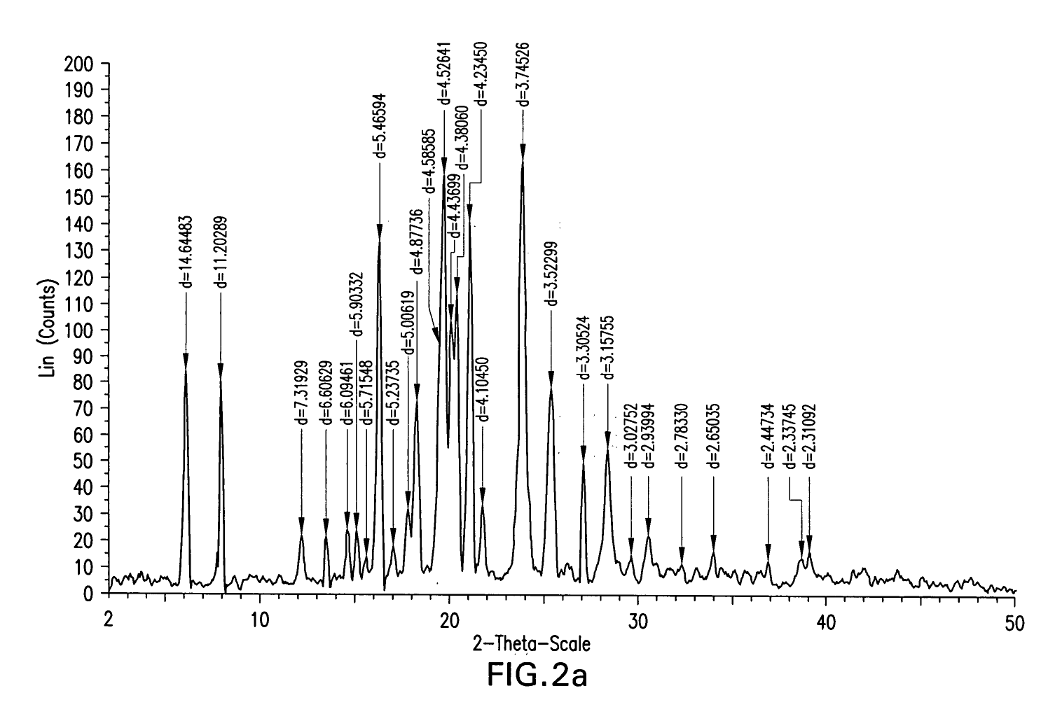

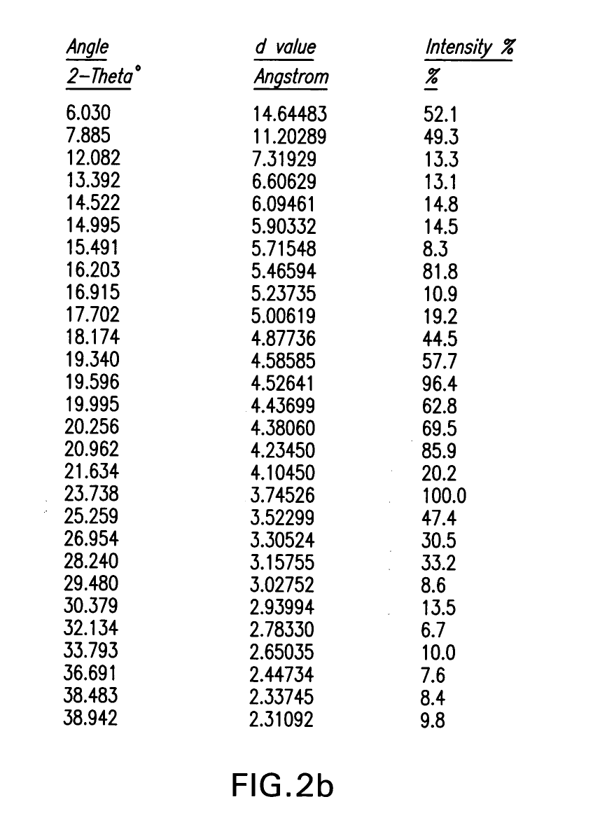

The invention also encompasses a process for preparing Compound 2a by crystallizing Compound 2a from a solvent comprising isopropanol, ethanol, and mixtures thereof, preferably using an antisolvent such as hexane or heptane. Preferably, the process produces a crystalline form of Compound 2a, denominated Compound 2a-Form 01, substantially characterized by PXRD patterns illustrated in FIGS. 1a, 2a, 3a or 4a. Preferably, the process produces crystalline Compound 2a having an enantiomeric purity of at least about 97.5%, more preferably at least about 98.5%, and most preferably at least about 99%.

The invention further encompasses a process for crystallizing Compound 2a by crystallizing Compound 2a from a solvent including toluene, ethanol, acetonitrile, methyl isobutyl ketone (MIBK), dichloromethane-hexane, methanol, acetone-water, or mixtures thereof. The preferred solvents are toluene, ethanol, or mixtures thereof. Preferably, the recrystallized Compound 2a is Compound 2a-Form 01. Preferably, the process is carried out after a first crystallization step.

The invention also encompasses Compound 2a prepared according to a process of the invention. Compound 2a prepared according to the invention may be used for the synthesis of ezetimibe by methods known in the art. Example 10 exemplifies one method of synthesizing ezetimibe from Compound 2a. Other synthetic pathways can be found, e.g., in the '171 patent, incorporated herein by reference.

The invention further encompasses a process for preparing ezetimibe comprising preparing Compound 2a according to a process of the invention, and converting Compound 2a to ezetimibe. Compound 2a may be converted to ezetimibe according methods known in the art, such as the process illustrated in Example 10 or in the '171 patent. The invention also encompasses ezetimibe prepared therefrom.

The invention encompasses a process for preparing ezetimibe comprising preparing Compound 2a-Form 01 according to a process of the invention, and converting Compound 2a-Form 01 to ezetimibe. The invention also encompasses ezetimibe prepared from any one process of the invention.

The present invention encompasses a process for preparing a compound of the formula:

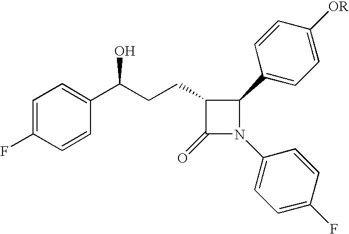

comprising combining a compound of the formula:

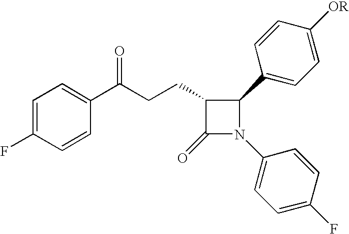

wherein R is H or a hydroxyl protecting group; a chiral catalyst; a hydrogen source including at least one of formic acid or a salt thereof, C₃-C₁₃ secondary alcohol, or cyclohexadiene; and an organic solvent, and recovering the product. Preferably, R is H.

Preferably, the hydroxyl protecting group is selected from the group consisting of benzyl and silyl. Examples of silyl protecting groups include (R^{a})(R^{b})(R^{c})—Si—, wherein R^{a}, R^{b} and R^{c} are the same or different and each are selected from the group consisting of C₁ to C₆ alkyl, phenyl, benzyl, or the like. Preferably, the silyl protecting group is selected from trimethylsilyl or tert-butyldimethylsilyl.

The chiral catalyst may be heterogeneous or homogeneous, and may include Ru catalysts with chiral ligands. Preferably, the chiral catalyst includes at least one catalyst selected from the group consisting of: [(S)-Xylyl-HexaPHEMP RuCl₂ (S,S)-DPEN], [(S)-HexaPHEMP RuCl₂ (S,S)-DACH], [(S)-HexaPHEMP RuCl₂ (S,S)-DPEN], [(R)-PhanePhos RuCl₂ (S,S)-DACH], [(R)-PhanePhos RuCl₂ (S,S)-DPEN], [(S)-MeO-Xylyl-PhanePhos RuCl₂ (R,R)-DPEN], [(R)-MeO-Xylyl-PhanePhos RuCl₂ (S,S)-DACH], [(S)-Tol-BINAP RuCl₂ (S,S)-DPEN], [(S)-SynPhos RuCl₂ (S,S)-DPEN], [(S)-Xylyl-BINAP RuCl₂ (S,S)-DPEN], [(R)-F-Phenyl-PhanePhos RuCl₂ (S,S)-DPEN], [(R)-MeO-Phenyl-PhanePhos RuCl₂ (S,S)-DPEN], [(R)-MeO-Phenyl-PhanePhos RuCl₂ (S,S)-DACH], [(R)-Xylyl-PhanePhos RuCl₂ (S,S)-DPEN], [(S,S)-Me-DuPhos RuCl₂ (S,S)-DPEN], (S,S)-TsDPEN Ru (p-cymene)Cl, [(S,S)-Me-DuPhos RuCl₂ (S,S)-DPEN], and [(S)-Tol-BINAP RuCl₂ (S,S)-DPEN].

Preferably, the C₃-C₁₃ secondary alcohol is isopropanol (IPA).

Optionally, a base may be added. Preferably, the base is an organic base. Preferably, the organic base includes at least one of triethylamine and tert-butoxide.

Preferably, the organic solvent is selected from the group consisting of: dichloromethane alcohols, THF, dioxane, and mixtures thereof. More preferably, the organic solvent is selected from the group consisting of dichloromethane, isopropanol, and mixtures thereof.

Preferably, the process for preparing a compound of the formula:

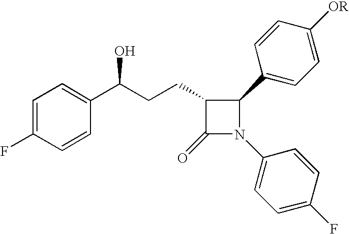

comprises combining a compound of the formula:

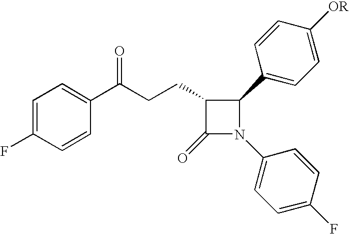

with a chiral catalyst and an organic solvent; adding a hydrogen source including at least one of formic acid or a salt thereof, isopropanol, or cyclohexadiene; stirring, and recovering the compound.

Preferably, prior to the addition of the hydrogen source, a solution is obtained.

Preferably, the hydrogen source is combined at a temperature of about 20° C. to about 40° C., and more preferably at a temperature of about 30° C. Preferably, the stirring is for about 10 to about 30 hours, more preferably about 19 hours.

Preferably, after stirring, the reaction mixture is analyzed by HPLC. Based on HPLC analysis, additional amount of a chiral catalyst and/or a hydrogen source may be added to the reaction mixture.

Preferably, after stirring, the reaction mixture is cooled to a temperature of about 30° C. to about 18° C., and more preferably about 25° C. to about 18° C. Preferably, the recovery comprises: adding a saturated aqueous sodium hydrogen carbonate solution to obtain a two phase system where the organic phase contains a precipitate, separating the phases, and extracting the precipitate from the organic phase. Preferably, the extraction comprises washing the organic layer with water, drying, filtering and concentrating to obtain a precipitate. Optionally, the precipitate is further crystallized from a solvent comprising at least one of acetonitrile, methyl isobutyl ketone, dichloromethane-hexane, acetone-water, ethanol-heptane, ethanol, toluene, or a C₁-C₆ alcohol and water mixture.

The invention encompasses a process for preparing a compound of the formula:

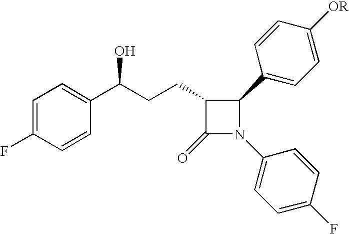

comprising: combining a compound of the formula:

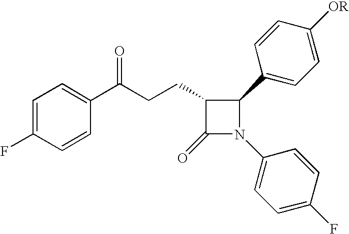

wherein R is H or a hydroxyl protecting group, and a chiral catalyst under an inert gas environment; adding an organic base to obtain a reaction mixture; subjecting the reaction mixture to a hydrogen pressure of about 4 bars to about 40 bars to produce the product; and recovering the product.

Preferably, the hydroxyl protecting group is selected from the group consisting of benzyl and silyl. Examples of silyl protecting groups include (R^{a})(R^{b})(R^{c})—Si—, wherein R^{a}, R^{b} and R^{c} are the same or different and each are selected from the group consisting of C₁ to C₆ alkyl, phenyl, benzyl, or the like. Preferably, the silyl protecting group is selected from trimethylsilyl or tert-butyldimethylsilyl.

Preferably, the inert gas is nitrogen. Preferably, the inert gas environment is maintained at a pressure of about 4 bars to about 15 bars, and more preferably at about 10 bars.

Preferably, after the organic base addition the reaction mixture is heated to a temperature of about 30° C. to about 45° C., and more preferably to about 40° C. Preferably, the heating is done while stirring.

Preferably, the hydrogen pressure is of about 4 bars to about 20 bars, and more preferably about 10 bars. Preferably, the hydrogen pressure is subsequently released. Preferably, the reaction is mixture cooled after the hydrogen pressure is released. Prior to cooling, the reaction mixture is preferably maintained for about 10 hours to about 30 hours, and more preferably for about 18 hours.

Preferably, the cooling is to a temperature of about 30° C. to about 18° C., and more preferably about 25° C. to about 18° C. Preferably, after cooling, a precipitate is formed.

Preferably, the recovery comprises concentrating and crystallizing the precipitate. Preferably, concentration is carried out under reduced pressure. Optionally, the precipitate is crystallized from a solvent comprising at least one of acetonitrile, methyl isobutyl ketone, dichloromethane-hexane, acetone-water, ethanol-heptane, ethanol, toluene, or a C₁-C₆ alcohol and water mixture.

The invention encompasses a pharmaceutical composition comprising ezetimibe prepared according to a process of the invention, and at least one pharmaceutically acceptable excipient.

The invention also encompasses a process for preparing a pharmaceutical composition comprising combining ezetimibe prepared according to a process of the invention with at least one pharmaceutically acceptable excipient.

The invention further encompasses use of ezetimibe prepared according to a process of the present invention for the manufacture of a pharmaceutical composition.

The invention also encompasses a method of reducing cholesterol comprising administering to a mammal in need thereof a composition of the invention.

Methods of administration of a pharmaceutical composition of the present invention can be administered in various preparations depending on the age, sex, and symptoms of the patient. The pharmaceutical compositions can be administered, for example, as tablets, pills, powders, liquids, suspensions, emulsions, granules, capsules, suppositories, injection preparations (solutions and suspensions), and the like.

Pharmaceutical compositions of the present invention can optionally be mixed with other forms of ezetimibe and/or other active ingredients such as HMG-CoA reductase inhibitors. In addition, pharmaceutical compositions of the present invention can contain inactive ingredients such as diluents, carriers, fillers, bulking agents, binders, disintegrants, disintegration inhibitors, absorption accelerators, wetting agents, lubricants, glidants, surface active agents, flavoring agents, and the like. Selection of excipients and the amounts to use can be readily determined by an experienced formulation scientist in view of standard procedures and reference works known in the art.

For example, diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid.

Solid and liquid compositions may also be dyed using any pharmaceutically acceptable colorant to improve their appearance and/or facilitate patient identification of the product and unit dosage level.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxyl toluene, butylated hydroxyanisole and ethylenediamine tetraacetic acid may be added at levels safe for ingestion to improve storage stability.

A liquid composition may also contain a buffer such as guconic acid, lactic acid, citric acid or acetic acid, sodium guconate, sodium lactate, sodium citrate or sodium acetate. Selection of excipients and the amounts used may be readily determined by the formulation scientist based upon experience and consideration of standard procedures and reference works in the field.

The solid compositions of the invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and losenges, as well as liquid syrups, suspensions and elixirs. The dosage form of the invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

The amount of ezetimibe or pharmaceutically acceptable salt thereof contained in a pharmaceutical composition for reducing cholesterol according to the present invention is not specifically restricted; however, the dose should be sufficient to treat, ameliorate, or reduce the condition. For example, ezetimibe may be present in an amount of about 1% to about 70%.

The dosage of a pharmaceutical composition for reducing cholesterol according to the present invention will depend on the method of use, the age, sex, weight and condition of the patient. Typically, about 1 mg to 200 mg of ezetimibe may be contained in an administration unit form, preferably a 10 mg tablet.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. Absent statement to the contrary, any combination of the specific embodiments described above are consistent with and encompassed by the present invention.

### EXAMPLES

All percentages are by area percent HPLC. The diastereoisomers (Compound 2a and Compound 2b) are separated using HPLC with the following parameters:

Column: Diacel Chiralcel OD-H, 5 micron, 250×4.6 mm
Eluent: heptane:ethanol (72:28)
Flow Rate: 0.3 ml/min

### Wavelength: 248 nm

Column temperature: 10° C.
Autosampler temperature: 10° C.
Diluent: ethanol

### Example 1

### Preparation of Compound 2a-Form 01

Into a 250 ml clean and dry 4 neck round bottom flask fitted with thermo pocket, N₂ gas inlet, guard tube and mechanical stirrer, 5 g (10.06 mmol) of (3R,4S)-4-((4-benzyloxy)phenyl)-1-(4-fluorophenyl)-3-(3-(4-fluorophenyl)-3-oxopropyl)-2-azetidinone and 50 ml of tetrahydrofuran were added at 25 to 30° C. The mixture was stirred at 25 to 30° C. until complete dissolution. To this solution 0.02 g (0.208 mmol) of methanesulfonic acid and 2.29 ml (2.2 mmol, 1 M solution in toluene) of (R)-tetrahydro-1-methyl-3,3-diphenyl-1H,3H-pyrrolo[1,2-C][1,3,2]oxazaborolidine were added. The mixture was cooled to −20 to −25° C., and 7.75 ml of borane dimethylsulfide complex (0.015 mol, 2M solution in THF) was added through an addition funnel over 30 min. The reaction mixture was stirred for 2 to 3 hrs at −20 to −25° C. and monitored by HPLC. After completion of the reaction, 5 ml of methanol was added, and the contents were stirred for 15-20 min. Then 5 ml of 1 N HCl was added, and the temperature was brought slowly to 10° C.

The reaction mixture was extracted with 50 ml of ethyl acetate. The aqueous layer was extracted again with 25 ml of ethyl acetate. The combined layers of ethyl acetate were washed with 2×50 ml of brine solution and then with 2×50 ml of water. The ethyl acetate layer was dried over sodium sulfate, and distilled and degassed under vacuum at 45 to 50° C. to produce (3R,4S)-4-((4-benzyloxy)phenyl)-1-(4-fluorophenyl)-3-((S)-3-(4-fluorophenyl)-3-hydroxypropyl)-2-azetidinone as an oil. The product was crystallized using ethanol/n-heptane, and recrystallized in toluene to yield 98.6% RSS isomer (Compound 2a-Form 01) and 1.4% RSR isomer (Compound 2b). See Tables 1 and 2. See also FIGS. 1a and 1b.

### Example 2

### Preparation of Compound 2a-Form 01

Into a 250 ml clean and dry 4 neck round bottom flask fitted with thermo pocket, N₂ gas inlet, guard tube and mechanical stirrer, 5.29 g (10.64 mmol) of (3R,4S)-4-((4-benzyloxy)phenyl)-1-(4-fluorophenyl)-3-(3-(4-fluorophenyl)-3-oxopropyl)-2-azetidinone and 50 ml of tetrahydrofuran were added at 25 to 30° C. The mixture was stirred at 25 to 30° C. until complete dissolution. To this solution 0.02 g (0.175 mmol) of trifluoroacetic acid and 2.4 ml (2.3 mmol, 1 M solution in toluene) of (R)-tetrahydro-1-methyl-3,3-diphenyl-1H,3H-pyrrolo[1,2-C][1,3,2]oxazaborolidine were added. The mixture was cooled to 15 to 20° C., and 6.0 ml of borane dimethylsulfide complex (0.012 mol, 2M solution in THF) was added by an addition funnel over 30 min. The reaction mixture was stirred for 2 to 3 hrs at 15 to 20° C. and monitored by HPLC. After completion of the reaction, 5 ml of methanol was added and the contents were stirred for 15-20 min. Then 5 ml of 1 N HCl was added, and the temperature was brought slowly to 10° C.

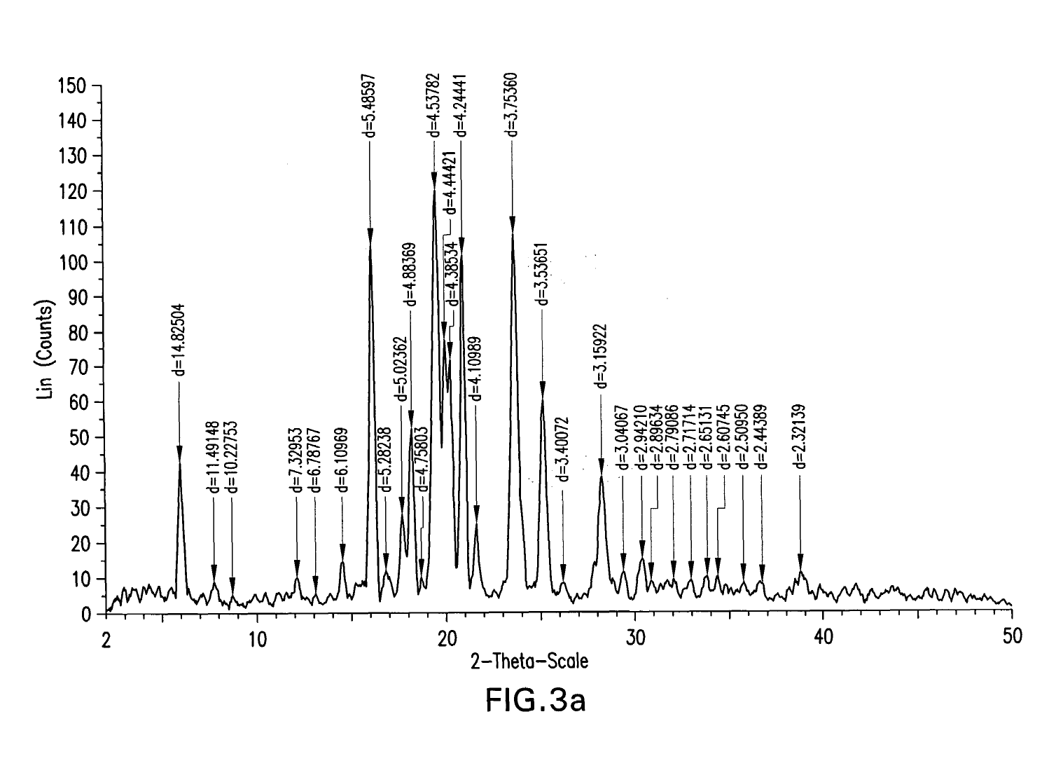

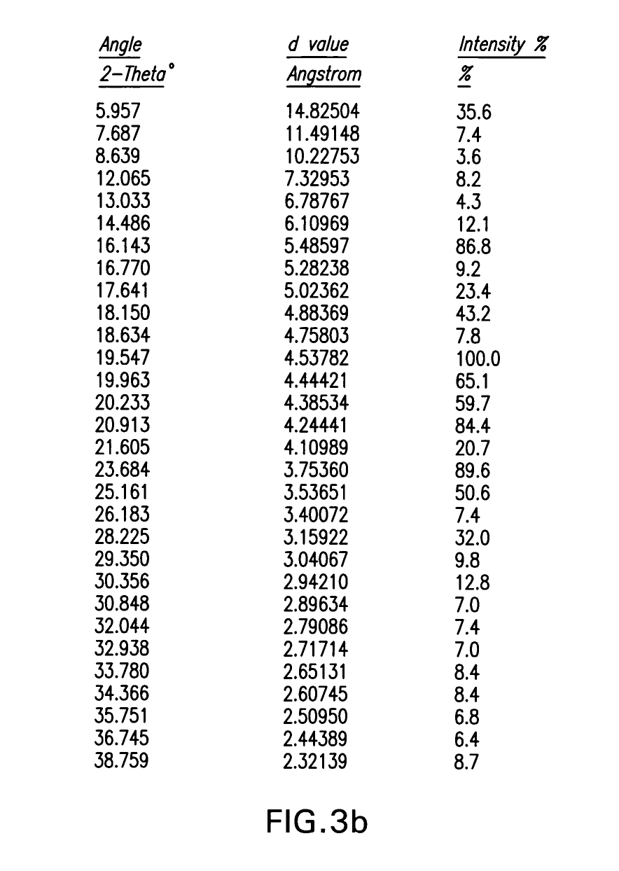

The reaction mixture was extracted with 50 ml of ethyl acetate. The aqueous layer was extracted again with 25 ml of ethyl acetate. The combined layers of ethyl acetate were washed with 2×50 ml of brine solution and then with 2×50 ml of water. The ethyl acetate layer was dried over sodium sulfate and distilled and degassed under vacuum at 45 to 50° C. to produce (3R,4S)-4-((4-benzyloxy)phenyl)-1-(4-fluorophenyl)-3-((S)-3-(4-fluorophenyl)-3-hydroxypropyl)-2-azetidinone as an oil. The product was crystallized using ethanol/n-heptane and recrystallized in toluene to yield 97.79% RSS isomer (Compound 2a-Form 01) and 2.21% RSR isomer (Compound 2b). See Tables 1 and 2. See also FIGS. 3a and 3b.

Into a 250 ml clean and dry 4 neck round bottom flask fitted with thermo pocket, N₂ gas inlet, guard tube and mechanical stirrer, 5.52 g (11.1 mmol) of (3R,4S)-4-((4-benzyloxy)phenyl)-1-(4-fluorophenyl)-3-(3-(4-fluorophenyl)-3-oxopropyl)-2-azetidinone and 50 ml of tetrahydrofuran were added at 25 to 30° C. The mixture was stirred at 25 to 30° C. until complete dissolution. To this solution 0.02 g (0.2 mmol) of methanesulfonic acid and 3.86 ml (3.8 mmol, 1 M solution in toluene) of (R)-tetrahydro-1-methyl-3,3-diphenyl-1H,3H-pyrrolo[1,2-C][1,3,2]oxazaborolidine were added. The mixture was cooled to −20 to −25° C., and 6.1 ml of borane dimethylsulfide complex (0.012 mol, 2M solution in THF) was added through an addition funnel over 30 min. The reaction mixture was stirred for 2 to 3 hrs at −20 to −25° C. and monitored by HPLC. After completion of the reaction, 6 ml of methanol was added and the contents were stirred for 15-20 min. Then 10 ml of 1 N HCl was added, and the temperature was brought slowly to 10° C.

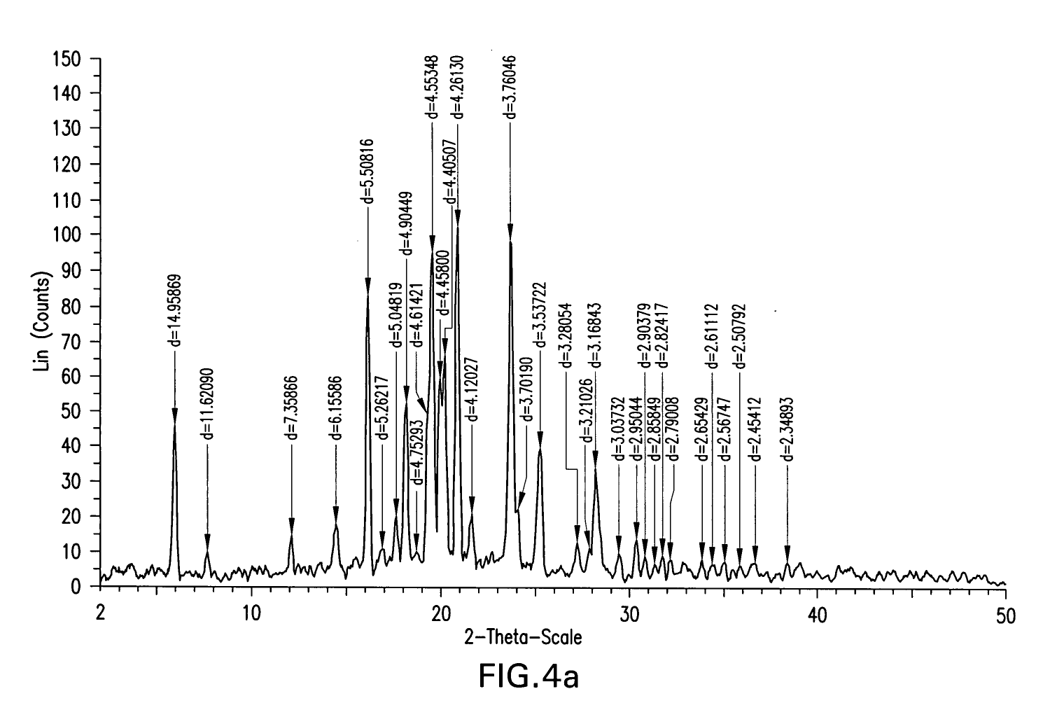

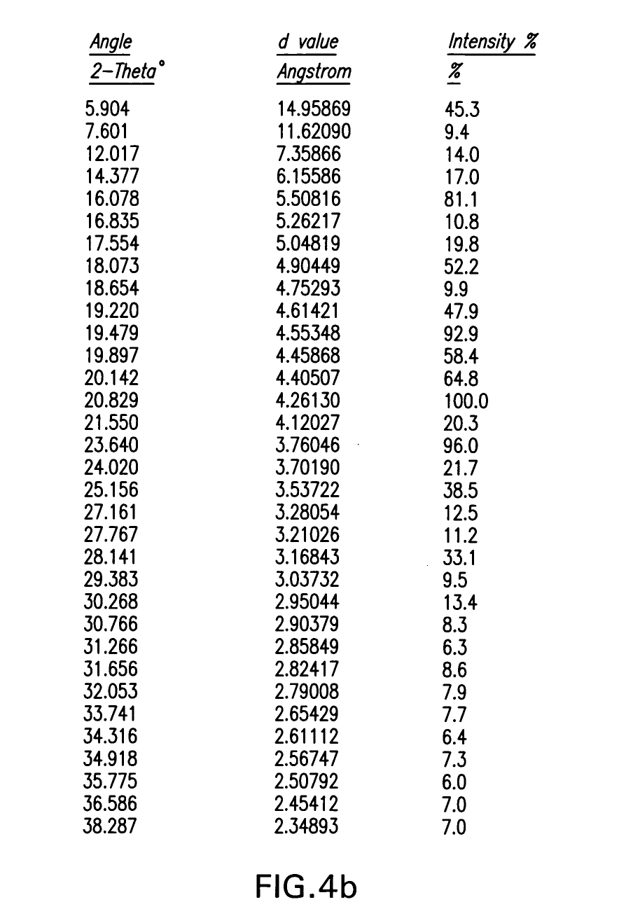

The reaction mixture was extracted with 50 ml of ethyl acetate. The aqueous layer was extracted again with 50 ml of ethyl acetate. The combined layers of ethyl acetate were washed with 2×50 ml of brine solution and then with 2×50 ml of water. The ethyl acetate layer was dried over sodium sulfate and distilled and degassed under vacuum at 45 to 50° C. to produce (3R,4S)-4-((4-benzyloxy)phenyl)-1-(4-fluorophenyl)-3-((S)-3-(4-fluorophenyl)-3-hydroxypropyl)-2-azetidinone as an oil. The product was crystallized using ethanol/n-heptane and recrystallized in ethanol to yield 98.73% RSS isomer (Compound 2a-Form 01) and 1.27% RSR isomer (Compound 2b). See Tables 1 and 2. See also FIGS. 4a and 4b.

### Example 4

### Preparation of Compound 2a-Form 01

Into a 250 ml clean and dry 4 neck round bottom flask fitted with thermo pocket, N₂ gas inlet, guard tube and mechanical stirrer, 5 g (10.06 mmol) of (3R,4S)-4-((4-benzyloxy)phenyl)-1-(4-fluorophenyl)-3-(3-(4-fluorophenyl)-3-oxopropyl)-2-azetidinone and 50 ml of tetrahydrofuran were added at 25 to 3° C. The mixture was stirred at 25 to 30° C. until complete dissolution. To this solution 0.02 g (0.208 mmol) of methanesulfonic acid and 2.29 ml (2.2 mmol, 1 M solution in toluene) of (R)-tetrahydro-1-methyl-3,3-diphenyl-1H,3H-pyrrolo[1,2-C][1,3,2]oxazaborolidine were added. The mixture was cooled to −20 to −25° C., and 15.0 ml of borane tetrahydrofuran complex (0.015 mol, 1M solution in THF) was added through an addition funnel over 30 min. The reaction mixture was stirred for 2 to 3 hrs at −20 to −25° C. and monitored by HPLC. After completion of the reaction, 5 ml of methanol was added and the contents were stirred for 15-20 min. Then 5 ml of 1 N HCl was added, and the temperature was brought slowly to 10° C.

The reaction mixture was extracted with 50 ml of ethyl acetate. The aqueous layer was extracted again with 25 ml of ethyl acetate. The combined layers of ethyl acetate were washed with 2×50 ml of brine solution and then with 2×50 ml of water. The ethyl acetate layer was dried over sodium sulfate and distilled and degassed under vacuum at 45 to 50° C. to produce (3R,4S)-4-((4-benzyloxy)phenyl)-1-(4-fluorophenyl)-3-((S)-3-(4-fluorophenyl)-3-hydroxypropyl)-2-azetidinone as an oil. The product was crystallized using ethanol/n-heptane, and recrystallized in ethanol to yield 98.65% RSS isomer (Compound 2a-Form 01) and 1.35% RSR isomer (Compound 2b). See Tables 1 and 2.

### Example 5

### Preparation of Compound 2a-Form 01

Into a 250 ml clean and dry 4 neck round bottom flask fitted with thermo pocket, N₂ gas inlet, guard tube and mechanical stirrer, 5.29 g (10.64 mmol) of (3R,4S)-4-((4-benzyloxy)phenyl)-1-(4-fluorophenyl)-3-(3-(4-fluorophenyl)-3-oxopropyl)-2-azetidinone and 50 ml of tetrahydrofuran were added at 25 to 30° C. The mixture was stirred at 25 to 30° C. until complete dissolution. To this solution 0.02 g (0.175 mmol) of trifluoroacetic acid and 2.4 ml (2.3 mmol, 1 M solution in toluene) of (R)-tetrahydro-1-methyl-3,3-diphenyl-1H,3H-pyrrolo[1,2-C][1,3,2]oxazaborolidine were added. The mixture was cooled to 15 to 20° C., and 12.0 ml of borane tetrahydrofuran complex (0.012 mol, 1M solution in THF) was added through an addition funnel over 30 min. The reaction mixture was stirred for 2 to 3 hrs at 15 to 20° C. and monitored by HPLC. After completion of the reaction, 5 ml of methanol was added and the contents were stirred for 15-20 min. Then 5 ml of 1 N HCl was added and the temperature was brought slowly to 10° C.

The reaction mixture was extracted with 50 ml of ethyl acetate. The aqueous layer was extracted again with 25 ml of ethyl acetate. The combined layers of ethyl acetate were washed with 2×50 ml of brine solution and then with 2×50 ml of water. The ethyl acetate layer was dried over sodium sulfate and distilled and degassed under vacuum at 45 to 50° C. to produce (3R,4S)-4-((4-benzyloxy)phenyl)-1-(4-fluorophenyl)-3-((S)-3-(4-fluorophenyl)-3-hydroxypropyl)-2-azetidinone as an oil. The product was crystallized using ethanol/n-heptane, and recrystallized in ethanol to yield 96.69% RSS isomer (Compound 2a-Form 01) and 3.31% RSR isomer (Compound 2b). See Tables 1 and 2.

### Example 6

### Preparation of Compound 2a-Form 01

Into a 250 ml clean and dry 4 neck round bottom flask fitted with thermo pocket, N₂ gas inlet, guard tube and mechanical stirrer was charged 6.53 g (0.013 mol) of (3R,4S)-4-((4-benzyloxy)phenyl)-1-(4-fluorophenyl)-3-(3-(4-fluorophenyl)-3-oxopropyl)-2-azetidinone and 70 ml of toluene were added at 25 to 30° C. The mixture was stirred at 25 to 30° C. until complete dissolution. To this solution 0.025 g (0.218 mmol) of methanesulfonic acid and 2.88 ml (2.8 mmol, 1 M solution in toluene) of (R)-tetrahydro-1-methyl-3,3-diphenyl-1H,3H-pyrrolo[1,2-C][1,3,2]oxazaborolidine were added. The mixture was cooled to −20 to −25° C., and 9.07 ml of borane dimethylsulfide complex (0.014 mol, 2M solution in THF) was added through an addition funnel over 30 min. The reaction mixture was stirred for 2-3 hrs at −20 to −25° C. and monitored by HPLC. After completion of the reaction, 6 ml of methanol was added at 0-5° C. and stirred for 15-20 min. Then 6 ml of 1 N HCl was added at 0-5° C.

The reaction mixture was extracted with 50 ml and 25 ml of ethyl acetate. The combined layers of ethyl acetate were washed with 2×50 ml of brine solution and then with 2×50 ml of water. The ethyl acetate layer was dried over sodium sulfate and distilled and degassed under vacuum at 45 to 50° C. to produce (3R,4S)-4-((4-benzyloxy)phenyl)-1-(4-fluorophenyl)-3-((S)-3-(4-fluorophenyl)-3-hydroxypropyl)-2-azetidinone as an oil. The product was crystallized using ethanol/n-heptane, and recrystallized in toluene to yield 97.36% RSS (Compound 2a-Form 01) isomer and 2.64% RSR isomer (Compound 2b). See Tables 1 and 2.

### Example 7

### Preparation of Compound 2a-Form 01

Into a 250 ml clean and dry 4 neck round bottom flask fitted with thermo pocket, N₂ gas inlet, guard tube and mechanical stirrer, 7.2 g (0.014 mol) of (3R,4S)-4-((4-benzyloxy)phenyl)-1-(4-fluorophenyl)-3-(3-(4-fluorophenyl)-3-oxopropyl)-2-azetidinone, 50 ml of tetrahydrofuran, and 50 ml of methyl tertiarybutylether were added at 25 to 30° C. The mixture was stirred at 25 to 30° C. until complete dissolution. To this solution 0.027 g (0.28 mmol) of methanesulfonic acid and 4.33 ml (4.3 mmol, 1 M solution in toluene) of (R)-tetrahydro-1-methyl-3,3-diphenyl-1H,3H-pyrrolo[1,2-C][1,3,2]oxazaborolidine were added. The mixture was cooled to −20 to −25° C., and 11.96 ml of borane dimethylsulfide complex (0.023 mol, 2M solution in THF) was added through an addition funnel over 30 min. The reaction mixture was stirred for 2-3 hrs at −20 to −25° C. and monitored by HPLC. After completion of the reaction, 6 ml of methanol was added at 0-5° C. and stirred for 15-20 min. Then 6 ml of 1 N HCl was added at 0-5° C.

The reaction mixture was extracted with 50 ml and 25 ml of ethyl acetate. The combined layers of ethyl acetate were washed with 2×50 ml of brine solution and then with 2×50 ml of water. The ethyl acetate layer was dried over sodium sulfate and distilled and degassed under vacuum at 45 to 50° C. to produce (3R,4S)-4-((4-benzyloxy)phenyl)-1-(4-fluorophenyl)-3-((S)-3-(4-fluorophenyl)-3-hydroxypropyl)-2-azetidinone as an oil. The product was crystallized using ethanol/n-heptane, and recrystallized in toluene to yield 98.04% RSS isomer (Compound 2a-Form 01) and 1.96% RSR isomer (Compound 2b). See Tables 1 and 2.

### Example 8

### Preparation of Compound 2a-Form 01

Into a 250 ml clean and dry 4 neck round bottom flask fitted with thermo pocket, N₂ gas inlet, guard tube and mechanical stirrer 3.0 g (6.0 mmol) of (3R,4S)-4-((4-benzyloxy)phenyl)-1-(4-fluorophenyl)-3-(3-(4-fluorophenyl)-3-oxopropyl)-2-azetidinone and 30 ml of tetrahydrofuran were added at 25 to 30° C. The mixture was stirred at 25 to 30° C. until complete dissolution. To this solution 0.017 g (0.11 mmol) of boron trifluoride etherate and 1.37 ml (1.37 mmol, 1M solution in toluene) of (R)-tetrahydro-1-methyl-3,3-diphenyl-1H,3H-pyrrolo[1,2-C][1,3,2]oxazaborolidine was added. The mixture was cooled to −20 to −25° C., and 3.3 ml of borane dimethylsulfide complex (6.6 mmol, 2M solution in THF) was added through an addition funnel in 30 min. The reaction mixture was stirred for 2-3 hrs at −20 to −25° C. and monitored by HPLC. After completion of the reaction, 6 ml of methanol was added at 0-5° C. and stirred for 15-20 min. Then 6 ml of 1 N HCl was added at 0-5° C.

The reaction mixture was extracted with 50 ml and 25 ml of ethyl acetate. The combined layers of ethyl acetate were washed with 2×50 ml of brine solution and then with 2×50 ml of water. The ethyl acetate layer was dried over sodium sulfate and distilled and degassed under vacuum at 45 to 50° C. to produce (3R,4S)-4-((4-benzyloxy)phenyl)-1-(4-fluorophenyl)-3-((S)-3-(4-fluorophenyl)-3-hydroxypropyl)-2-azetidinone as an oil. The product was crystallized using ethanol/n-heptane, and recrystallized in toluene to yield 96.3% RSS isomer (Compound 2a-Form 01) and 3.7% RSR isomer (Compound 2b). See Tables 1 and 2.

### Comparative Example 9

### Preparation of Compound 2a-Form 01

The following example was based largely on U.S. Pat. No. 5,631,365, incorporated herein by reference in its entirety.

Into a 250 ml clean and dry 4 neck round bottom flask fitted with thermo pocket, N₂ gas inlet, guard tube and mechanical stirrer was charged 5 g (10.06 mmol) of (3R,4S)-4-((4-benzyloxy)phenyl)-1-(4-fluorophenyl)-3-(3-(4-fluorophenyl)-3-oxopropyl)-2-azetidinone and 50 ml of tetrahydrofuran were added at 25 to 30° C. The mixture was stirred at 25 to 30° C. until complete dissolution. To this solution 2.29 ml (2.2 mmol, 1 M solution in toluene) of (R)-tetrahydro-1-methyl-3,3-diphenyl-1H,3H-pyrrolo[1,2-C][1,3,2]oxazaborolidine was added. The mixture was cooled to −20 to −25° C. and 7.75 ml of borane dimethylsulfide complex (0.015 mol, 2M solution in THF) was added through an addition funnel over 30 min. The reaction mixture was stirred for 2 to 3 hrs at −20 to −25° C. and monitored by HPLC. After completion of reaction, 5 ml of methanol was added and the contents were stirred for 15-20 min. Then 5 ml of 1 N HCl was added, and the temperature was brought slowly to 10° C.

The reaction mixture was extracted with 50 ml of ethyl acetate. The aqueous layer was extracted again with 25 ml of ethyl acetate. The combined layers of ethyl acetate were washed with 2×50 ml of brine solution and then with 2×50 ml of water. The ethyl acetate layer was dried over sodium sulfate and distilled and degassed under vacuum at 45 to 50° C. to product (3R,4S)-4-((4-benzyloxy)phenyl)-1-(4-fluorophenyl)-3-((S)-3-(4-fluorophenyl)-3-hydroxypropyl)-2-azetidinone as an oil.

The product was crystallized using ethanol/n-heptane, and recrystallized in toluene to yield 89.6% RSS isomer (Compound 2a-Form 01) and 10.4% RSR isomer (Compound 2b). See Table 1.

### Example 10

### Conversion of Compound 2a into Ezetimibe

Into a 500 ml SS parr shaker autoclave 10 g (0.02 mol) (3R,4S)-4-((4-benzyloxy)phenyl)-1-(4-fluorophenyl)-3-((S)-3-(4-fluorophenyl)-3-hydroxypropyl)-2-azetidinone, 150 ml of ethanol, and 3.0 g of 10% palladium on carbon (50%, wet) were added at room temperature. The autoclave was closed and flushed with nitrogen gas twice and pressurized with hydrogen gas to obtain a pressure of 5 kg/cm². The shaker was started and maintained for 6 hrs filling hydrogen gas up to 5 kg/cm² when required. The reaction was monitored by TLC, mobile phase, with ethylacetate:hexane (1:1). After completion of the reaction, the hydrogen gas was discharged, the reaction mixture flushed with nitrogen gas, and the catalyst was filtered under nitrogen. The solvent was distilled under reduced pressure, and the crude product was crystallized using isopropanol/water to produce ezetimibe.

### Example 11

### Preparation of Compound 2a-Form 01

Into a 3 L clean and dry 4 neck round bottom flask fitted with thermo pocket, N₂ gas inlet, guard tube and mechanical stirrer, 66.6 g (0.134 mol) of (3R,4S)-4-((4-benzyloxy)phenyl)-1-(4-fluorophenyl)-3-(3-(4-fluorophenyl)-3-oxopropyl)-2-azetidinone, and 666 ml of tetrahydrofuran were added at 25 to 30° C. The mixture was stirred at 25 to 30° C. until complete dissolution. To this solution 0.257 g (0.0026 mol) of methanesulfonic acid and 30.4 ml (0.030 mol, 1 M solution in toluene) of (R)-tetrahydro-1-methyl-3,3-diphenyl-1H,3H-pyrrolo[1,2-C][1,3,2]oxazaborolidine were added. The mixture was cooled to −20 to −25° C., and 94.8 ml of borane dimethylsulfide complex (0.186 mol, 2M solution in THF) was added through an addition funnel over 30 min. The reaction mixture was stirred for 2 to 3 hrs at −20 to −25° C. and monitored by HPLC. After completion of the reaction, 66.6 ml of methanol was added, and the contents were stirred for 15-20 min. Then 66.6 ml of 1 N HCl was added, and the temperature was brought slowly to 10° C.

The reaction mixture was extracted with 666 ml of ethyl acetate. The aqueous layer was extracted again with 335 ml of ethyl acetate. The combined layers of ethyl acetate were washed with 2×665 ml of brine solution and then with 2×665 ml of water. The ethyl acetate layer was dried over sodium sulfate, and distilled and degassed under vacuum at 45 to 50° C. to produce (3R,4S)-4-((4-benzyloxy)phenyl)-1-(4-fluorophenyl)-3-((S)-3-(4-fluorophenyl)-3-hydroxypropyl)-2-azetidinone as an oil. The product was crystallized using ethanol/n-heptane, and recrystallized in toluene to yield 99.4% RSS isomer (Compound 2a-Form 01) and 0.6% RSR isomer (Compound 2b). See Tables 1 and 2.

Table 1 illustrates the reaction conditions of Examples 1-9 and 11.

TABLE 1
Reduction of Compound 1 and Enantiomeric Purity

| | | | | | | Compound 2a | Compound 2b |
|---|---|---|---|---|---|---|---|
| | | | Reduction | | | (RSS) | (RSR) |
| | Reaction | | Temp. | Reducing | Chiral | First/Second | First/Second |
| Ex. | Solvent | Acid | (° C.) | Agent | Catalyst | Crystallization | Crystallization |
| 1 | THF | MSA | −25 to −20 | BH₃-Me₂S | (R)-Me-CBS | 98.4/98.6 | 1.6/1.4 |
| 2 | THF | TFA | 15 to 20 | BH₃-Me₂S | (R)-Me-CBS | 97.7/97.8 | 2.3/2.2 |
| 3 | THF | MSA | −25 to −20 | BH₃-Me₂S | (R)-Me-CBS | 98.7/98.7 | 1.3/1.3 |
| 4 | THF | MSA | −25 to −20 | BH₃-THF | (R)-Me-CBS | 98.6/98.7 | 1.4/1.3 |
| 5 | THF | TFA | 15 to 20 | BH₃-THF | (R)-Me-CBS | 97.7/96.7 | 2.3/3.3 |
| 6 | Toluene | MSA | −25 to −20 | BH₃-Me₂S | (R)-Me-CBS | 97.2/97.4 | 2.8/2.6 |
| 7 | THF: | MSA | −25 to −20 | BH₃-Me₂S | (R)-Me-CBS | 97.7/98.0 | 2.3/2.0 |
| | MTBE | | | | | | |
| 8 | THF | BF₃OEt₂ | −25 to −20 | BH₃-Me₂S | (R)-Me-CBS | 97.9/96.3 | 2.1/3.7 |
| 9 | THF | — | −25 to −20 | BH₃-Me₂S | (R)-Me-CBS | NA/89.6 | NA/10.4 |
| 11 | THF | MSA | −25 to −20 | BH₃-Me₂S | (R)-Me-CBS | 99.4/99.4 | 0.6/0.6 |

MSA = methanesulfonic acid.
TFA = trifluoroacetic acid.
BF₃OEt₂ = boron trifluoride etherate.

Table 2 illustrates the enantiomeric excess, chemical purity, and yield of Compound 2a from Examples 1-8 and 11. Enantiomeric excess is calculated as follows:

e.e.=(RSS−RSR)/(RSS+RSR)×100%.

TABLE 2
Compound 2a

| | First Crystallization | | | Second Crystallization | | |
|---|---|---|---|---|---|---|
| Example | Enantiomeric | Chemical | Overall | Enantiomeric | Chemical | Overall |
| No | excess | Purity | Yield | excess | Purity | Yield |
| 1 | 96.8% | 94.2% | 65.8% | 97.2% | 99.0% | 49.4% |
| 2 | 95.4% | 94.4% | 61.3% | 95.6% | 98.1% | 47.6% |
| 3 | 97.4% | 94.6% | 67.8% | 97.5% | 98.5% | 47.6% |
| 4 | 97.24% | 94.1% | 63.8% | 97.3% | 98.3% | 46.6% |
| 5 | 95.4% | 94.3% | 68.3% | 93.4% | 97.2% | 52.4% |
| 6 | 94.4% | 74.0% | 62.4% | 94.7% | 90.0% | 40.3% |
| 7 | 95.4% | 88.0% | 68.9% | 96.1% | 92.0% | 51.2% |
| 8 | 95.8% | 92.0% | 61.7% | 92.6% | 97.2% | 48.2% |
| 11 | 98.7% | 91.4% | 58.6% | 98.8% | 99.0% | 50.4% |

### Example 12

### Transfer Hydrogenation

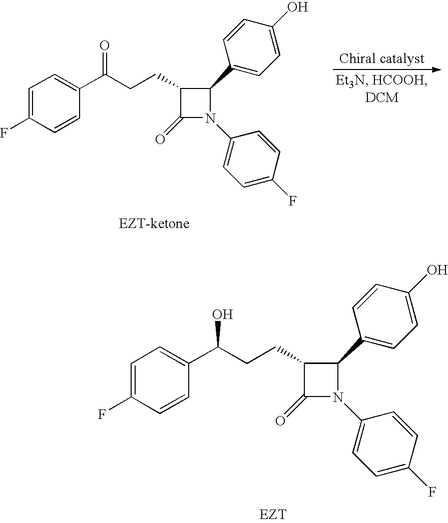

Formic acid (7.2 mL, 190.7 mmol) is added dropwise to a stirred solution of ezetimibe-ketone (15.6 g, 38.5 mmol), (S,S)-TsDPEN Ru (p-cymene)Cl (231 mg, 0.36 mmol) and triethylamine (26 mL, 186.5 mmol) in dichloromethane (50 mL) at 30° C. (internal) under nitrogen atmosphere over a period of 30 minutes. The internal temperature reaches 35° C. during the addition. After stirring for 19 hours at 30° C., the reaction is followed by HPLC analysis and based on the results additional (S,S)-TsDPEN Ru (p-cymene)Cl (47 mg, 0.07 mmol) is added to the reaction mixture, followed by formic acid (3 mL, 79.5 mmol) added dropwise over 30 minutes. After stirring for 21 hours at 35° C. (internal), the reaction is allowed to cool to room temperature, and saturated aqueous sodium hydrogen carbonate solution (100 mL) is added. The two layers are then separated and the aqueous layer is further extracted with dichloromethane (80 mL). The combined organic layers are washed with water (80 mL), dried (MgSO₄), filtered and concentrated under reduced pressure. The crude material is purified by crystallization (aqueous IPA).

### Example 13

### Transfer Hydrogenation

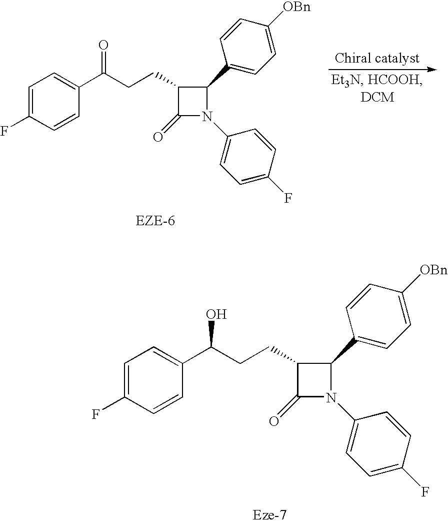

Formic acid (7.2 mL, 190.7 mmol) is added dropwise to a stirred solution of Eze-6 (19.1 g, 38.5 mmol), (S,S)-TsDPEN Ru (p-cymene)Cl (231 mg, 0.36 mmol) and triethylamine (26 mL, 186.5 mmol) in dichloromethane (50 mL) at 30° C. (internal) under a nitrogen atmosphere over a period of 30 minutes. The internal temperature reaches 35° C. during the addition. After stirring for 19 hours at 30° C., the reaction is followed by HPLC analysis and based on the results additional (S,S)-TsDPEN Ru (p-cymene)Cl (47 mg, 0.07 mmol) is added to the reaction mixture, followed by formic acid (3 mL, 79.5 mmol) added dropwise over 30 minutes. After stirring for 21 hours at 35° C. (internal), the reaction is allowed to cool to room temperature, and saturated aqueous sodium hydrogen carbonate solution (100 mL) is added. The two layers are then separated and the aqueous layer is further extracted with dichloromethane (80 mL). The combined organic layers are washed with water (80 mL), dried (MgSO₄), filtered and concentrated under reduced pressure. The crude material is purified by crystallization (ethanol).

### Example 14

### Hydrogenation

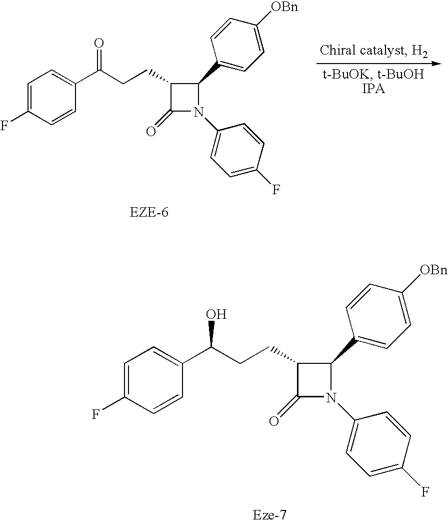

[(S,S)-Me-DuPhos RuCl₂ (S,S)-DPEN] (1.7 mg, 0.002 mmol) and Eze-6 (250 mg, 0.5 mmol) are placed in a glass liner within an Argonaut Endeaver pressure vessel. The vessel is assembled and pressurized to 10 bar with nitrogen and the pressure is released. The procedure is repeated a twice. A solution of potassium tert-butoxide [3 ml (of a solution of commercial 0.25 ml of 1M potassium tert-butoxide solution in butanol made up to 30 ml with dry degassed 2-propanol), 0.025 mmol] is added to the vessel. The vessel is pressurized to 10 bar with nitrogen and the pressure is released. The vessel is heated to 40° C. (internal) with stirring before being pressurized to 10 bar with hydrogen. After 18 hours, the vessel is allowed to cool to room temperature before being vented, and the reaction solution is concentrated under reduced pressure to afford Eze-7 which is purified by crystallization (ethanol).

### Example 15

### Hydrogenation

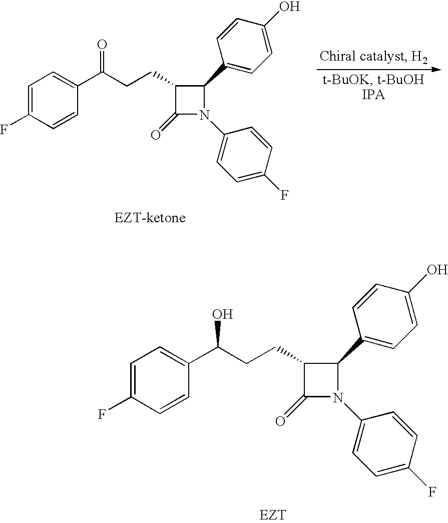

[(S)-Tol-BINAP RuCl2 (S,S)-DPEN] (1.7 mg,) and EZT-ketone (250 mg) are placed in a glass liner within an Argonaut Endeaver pressure vessel. The vessel is assembled and pressurized to 10 bar with nitrogen and the pressure is released. The procedure is repeated twice. A solution of potassium tert-butoxide [3 ml (of a solution of commercial 0.25 ml of 1M potassium tert-butoxide solution in butanol made up to 30 ml with dry degassed 2-propanol), 0.025 mmol] is added to the vessel. The vessel is pressurized to 10 bar with nitrogen and the pressure is released. The vessel is heated to 40° C. (internal) with stirring before being pressurized to 10 bar with hydrogen. After 18 hours, the vessel is allowed to cool to room temperature. Ezetimibe is isolated by addition of water (3 ml).

1. (3R,4S)-4-((4-benzyloxy)phenyl)-1-(4-fluorophenyl)-3-(3-(4-fluorophenyl-)-3-oxopropyl)-2-azetidinone (Compound 2a) of the following formula: 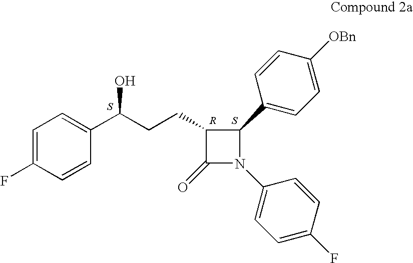
having a purity profile selected from at least one of (i) an enantiomeric purity of at least about 97.5%, (ii) having less than about 2.5% by area percent HPLC of Compound 2b having the following formula: 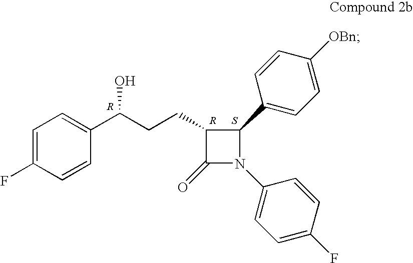
and (iii) having a chemical purity of at least about 97% by area percent HPLC.

2-8. (canceled)

9. A process for preparing Compound 2a comprising combining (3R,4S)-4-((4-benzyloxy)-phenyl)-1-(4-fluorophenyl)-3-(3-(4-fluorophenyl)-3-oxopropyl)-2-azetidinone (Compound 1): 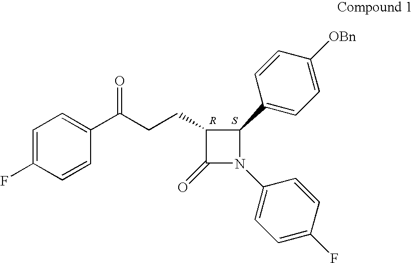
and a solvent selected from the group consisting of a cyclic ether, ether, halogenated hydrocarbon, aromatic hydrocarbon, and mixtures thereof to obtain a solution; adding an acid, a chiral catalyst, and a sufficient amount of a borane reducing agent to obtain Compound 2a; and recovering Compound 2a.

10. The process of claim 9, wherein the acid is selected from the group consisting of methanesulfonic acid, trifluoroacetic acid, boron trifluoride etherate, and mixtures thereof.

11. The process of claim 9, wherein the acid is methanesulfonic acid.

12. The process of claim 9, wherein the ratio of the acid to Compound 1 is in a molar % of about 1% to about 5%.

13. The process of claim 9, wherein the ratio of the acid to Compound 1 is in a molar % of about 1.6% to about 2%.

14. The process of claim 9, wherein the solvent is selected from the group consisting of tetrahydrofuran, toluene, dichloromethane, 2-methyl THF, methyl tert butyl ether, and mixtures thereof.

15. The process of claim 9, wherein the solvent includes tetrahydrofuran.

16. The process of claim 9, wherein the chiral catalyst includes at least one of (R)-tetrahydro-1-methyl-3,3-diphenyl-1H,3H-pyrrolo[1,2-C][1,3,2]ox-azaborolidine, or (R)-tetrahydro-1-phenyl-3,3-diphenyl-1H,3H-pyrrolo[1,2-C][1,3,2]oxazaborolidine.

17. The process of claim 9, wherein the chiral catalyst includes (R)-tetrahydro-1-methyl-3,3-diphenyl-1H,3H-pyrrolo[1,2-C][1,3,2]oxazaborolidine.

18. The process of claim 9, wherein the chiral catalyst is added at a temperature of about 25° C. to about 30° C.

19. The process of claim 9, wherein the ratio of the chiral catalyst to Compound 1 is in a molar percentage of about 20% to about 40%.

20. The process of claim 9, wherein the borane reducing agent is selected from the group consisting of a borane-tetrahydrofuran complex, borane-dimethylsulfide complex, borane 1,4-dioxane, borane diethylaniline, borane N-ethyl-N-isopropylaniline, N-borane phenylamine, catecholborane, in situ generated borane, and mixtures thereof.

21. The process of claim 9, wherein the borane reducing is selected from the group consisting of a borane-tetrahydrofuran complex, borane-dimethylsulfide complex, and mixtures thereof.

22. The process of claim 9, wherein the ratio of the borane reducing agent to Compound 1 is in a molar percentage of about 100% to about 200%.

23. The process of claim 9, wherein the ratio of the borane reducing agent to Compound 1 is in a molar percentage of about 100% to about 170%.

24. The process of any claim 9, wherein the borane reducing agent is added at a temperature of about −30° C. to about −15° C.

25. The process of claim 9, wherein the borane reducing agent is added at a temperature of about −25° C. to about −20° C.

26. The process of claim 9, wherein a reaction mixture containing Compound 2a is obtained before recovering Compound 2a.

27. The process of claim 9, wherein a reaction mixture containing Compound 2a is obtained before recovering Compound 2a, and the reaction mixture is stirred.

28. The process of claim 9 wherein a reaction mixture containing Compound 2a is obtained before recovering Compound 2a, and the reaction mixture is stirred at a temperature of about 0° C. to about 15° C.

29. The process of claim 9, wherein a reaction mixture containing Compound 2a is obtained before recovering Compound 2a; and the recovering comprises quenching the reaction mixture with a solvent selected from the group consisting of methanol, acetone, and mixtures thereof, and extracting Compound 2a.

30. The process of claim 9, wherein prior to the extraction, an acid suitable to decompose the excess borane complex is added.

31. The process of claim 9, wherein a reaction mixture containing Compound 2a is obtained before recovering Compound 2a; and the reaction mixture is extracted with ethyl acetate and water to recover Compound 2a.

32. The process of claim 9, wherein the process produces Compound 2a having an enantiomeric purity of at least about 97.5%.

33. The process of claim 9, wherein the process produces Compound 2a having an enantiomeric purity of at least about 98.5%.

34. The process of claim 9, wherein the process produces Compound 2a having a chemical purity of at least about 97% by area percent HPLC.

35. The process of claim 9, further comprising crystallizing Compound 2a from a solvent comprising isopropanol, ethanol, and mixtures thereof, using an antisolvent including at least one of hexane or heptane.

36. A process for preparing (3R,4S)-4-((4-benzyloxy)phenyl)-1-(4-fluorophenyl)-3-(3-(4-fluorophenyl)-3-oxopropyl)-2-azetidinone (Compound 2a) comprising crystallizing Compound 2a from a solvent comprising isopropanol, ethanol, and mixtures thereof, using an antisolvent including at least one of hexane or heptane.

37. The process of claim 36, further comprising recrystallizing the Compound 2a in a recrystallization solvent selected from the group consisting of toluene, ethanol, acetonitrile, MIBK, dichloromethane-hexane, methanol, acetone-water, ethanol-heptane, and mixtures thereof.

38. A process for preparing (3R,4S)-4-((4-benzyloxy)phenyl)-1-(4-fluorophenyl)-3-(3-(4-fluorophenyl)-3-oxopropyl)-2-azetidinone (Compound 2a) comprising crystallizing Compound 2a in a crystallization solvent selected from the group consisting of toluene, ethanol, acetonitrile, MIBK, dichloromethane-hexane, methanol, acetone-water, ethanol-heptane, and mixtures thereof.

39. The process of claim 38, wherein the Compound 2a obtained is Compound 2a-Form 01.

40-41. (canceled)

42. A process for preparing a compound of the formula: 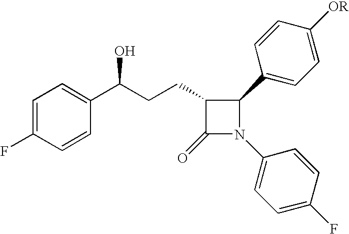
comprising combining a compound of the formula: 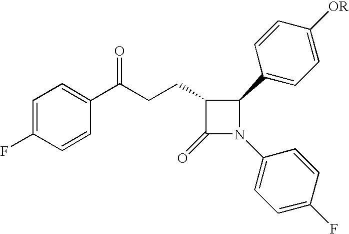
wherein R is H or a hydroxyl protecting group; a chiral catalyst; a hydrogen source including at least one of formic acid or a salt thereof, C₃-C₁₃ secondary alcohol, or cyclohexadiene; and an organic solvent, and recovering the product.

43. (canceled)

44. The process of claim 42, wherein the chiral catalyst is selected from the group consisting of [(S)-Xylyl-HexaPHEMP RuCl₂ (S,S)-DPEN], [(S)-HexaPHEMP RuCl₂ (S,S)-DACH], [(S)-HexaPHEMP RuCl₂ (S,S)-DPEN], [(R)-PhanePhos RuCl₂ (S,S)-DACH], [(R)-PhanePhos RuCl₂ (S,S)-DPEN], [(S)-MeO-Xylyl-PhanePhos RuCl₂ (R,R)-DPEN], [(R)-MeO-Xylyl-PhanePhos RuCl₂ (S,S)-DACH], [(S)-Tol-BINAP RuCl₂ (S,S)-DPEN], [(S)-SynPhos RuCl₂ (S,S)-DPEN], [(S)-Xylyl-BINAP RuCl₂ (S,S)-DPEN], [(R)-F-Phenyl-PhanePhos RuCl₂ (S,S)-DPEN], [(R)-MeO-Phenyl-PhanePhos RuCl₂ (S,S)-DPEN], [(R)-MeO-Phenyl-PhanePhos RuCl₂ (S,S)-DACH], [(R)-Xylyl-PhanePhos RuCl₂ (S,S)-DPEN], [(S,S)-Me-DuPhos RuCl₂ (S,S)-DPEN], (S,S)-TsDPEN Ru p-cymene)Cl, [(S,S)-Me-DuPhos RuCl₂ (S,S)-DPEN], [(S)-Tol-BINAP RuCl₂ (S,S)-DPEN], and mixtures thereof.

45. (canceled)

46. The process of claim 42, further comprising adding an inorganic base.

47-49. (canceled)

50. The process of claim 42, wherein the process comprises combining a compound of the formula: 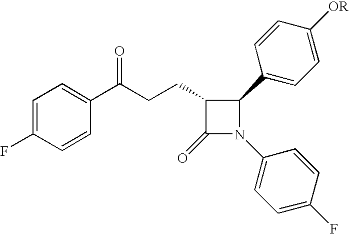
with a chiral catalyst and an organic solvent to obtain a solution; adding a hydrogen source selected from at least one of formic acid or a salt thereof, isopropanol, or cyclohexadiene; stirring; and recovering the product.

51-53. (canceled)

54. A process for preparing a compound of the formula: 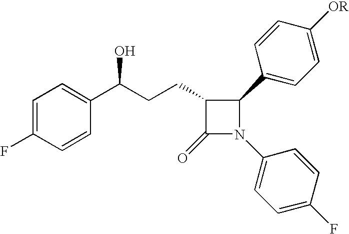
comprising: combining a compound of the formula: 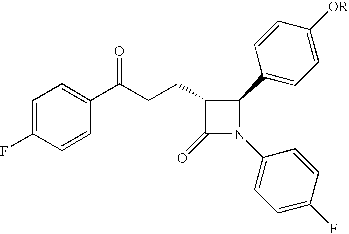
wherein R is H or a hydroxyl protecting group, and a chiral catalyst under an inert gas environment; adding an organic base to obtain a reaction mixture; subjecting the reaction mixture to a hydrogen pressure of about 4 bars to about 40 bars to produce the product; and recovering the product.

55-65. (canceled)

66. The process of claim 54, wherein the recovering includes crystallizing from a solvent selected from at least one of ethanol, toluene, or a C₁-C₆ alcohol and water mixture.

67. (canceled)

68. The process of claim 54, further comprising crystallizing Compound 2a from a solvent comprising isopropanol, ethanol, and mixtures thereof, using an antisolvent including at least one of hexane or heptane.

69. The process of claim 54, further comprising crystallizing in a crystallization solvent selected from the group consisting of toluene, ethanol, acetonitrile, MIBK, dichloromethane-hexane, methanol, acetone-water, ethanol-heptane, and mixtures thereof.

70. The process of claim 54, wherein the Compound 2a obtained is Compound 2a-Form 01.

71. (canceled)

72. Compound 2a prepared by the process of claim 9.

73. A process for preparing ezetimibe comprising converting the Compound 2a of claim 1 to ezetimibe.

74. A process for preparing ezetimibe comprising converting Compound 2a prepared by the process of claim 9 to ezetimibe.

75. Compound 2a-Form 01 prepared by the process of claim 39.

76. Ezetimibe prepared by the process of claim 73.

77. A pharmaceutical composition comprising the ezetimibe of claim 76 and at least one pharmaceutically acceptable excipient.

78. A process for preparing a pharmaceutical formulation comprising combining the ezetimibe of claim 76 with at least one pharmaceutically acceptable excipient.

79. The use of the ezetimibe of claim 76 for the manufacture of a pharmaceutical composition.

80. A method of reducing cholesterol comprising administering to a mammal in need thereof the composition of claim 77.

81. Use of the composition of claim 77 in the manufacture of a medicament for reducing cholesterol.
